(19) United States
(12) Patent Application Publication
(10) Pub. No.: US 2011/0020157 A1
(43) Pub. Date: Jan. 27, 2011

(54) FEED PUMP AND SEALING ARRANGEMENT FOR SAME

(75) Inventor: Andreas Schmidt, Mutterstadt (DE)

Correspondence Address: SENNIGER POWERS LLP, 100 NORTH BROADWAY, 17TH FLOOR, ST LOUIS, MO, 63102, US

(73) Assignee: LINCOLN GMBH, Walldorf (DE)

(21) Appl. No.: 12/296,637

(22) PCT Filed: Mar. 20, 2007
(86) PCT No.: PCT/EP07/02430

§ 371 Date: Aug. 20, 2009

(30) Foreign Application Priority Data
Apr. 13, 2006 (DE) 20 2006 00 221.1

(51) Int. Cl. A61M 1/00; F16J 15/18

(52) U.S. Cl. 417/437; 92/165 R

(57) ABSTRACT

The present invention relates to a feed pump, in particular for pumping UV paints and other high-viscosity media, with a pump housing to which an inlet valve and an outlet valve are fitted, and a piston, which for pumping a medium is mounted in the pump housing in a manner as to permit positional shifting. To improve the seal and the piston lifetime, the piston is guided in at least one section of the pump housing by way of at least two seals positioned one behind the other in the axial direction of the piston so as to seal it from its surroundings and forming between them an intermediate gapped space that connects to the flow from a coolant and/or lubricant container.

### FIELD OF THE INVENTION

The invention pertains to a feed pump with a pump housing, to which an inlet valve and an outlet valve are assigned, and with a piston that is displaceably arranged in the pump housing in order to pump a medium. The invention further pertains to a sealing arrangement for a feed pump of this type.

### BACKGROUND OF THE INVENTION

Feed pumps are used, among other things, for pumping UV paints and other high-viscosity media. When pumping such media that, for example, harden at high temperatures or when they come in contact with other media, e.g., due to polymerization, the sealing arrangement between the piston and the pump housing is subject to particularly strict requirements. For example, friction between the piston and the pump housing needs to be largely prevented in order to prevent the polymerization, for example, of UV paints under elevated temperatures that are caused by friction. Feed pumps with conventional sealing arrangements are only conditionally suitable, in particular, for pumping UV paints and similar media with strong polymerization tendencies.

In addition, the service lives of known sealing arrangements for such operating conditions are usually not very long, so the seals need to be frequently replaced. This results in long standstill times for the repair of the pump. In some known feed pumps and sealing arrangements, one also has to accept the risk of the pumped media hardening on the piston of the feed pump and/or the sealing arrangement, even if friction is minimized.

### SUMMARY OF THE INVENTION

The present invention relates to a feed pump, in particular a pump for pumping UV paints and other high-viscosity media. The pump comprises a pump housing having an inlet valve and an outlet valve, and a piston that is displaceably arranged in the pump housing to pump a medium. The piston is guided in at least a section of the pump housing such that it is sealed relative to the surroundings by at least two seals that are arranged one behind the other in the axial direction of the piston. An intermediate space is formed between the at least two seals and is fluidically connected to a coolant and/or lubricant container.

The present invention is also directed to a sealing arrangement for a feed pump. The sealing arrangement comprises a piston guided in a sleeve-like component. At least two seals are arranged in the sleeve-like component and spaced apart from one another in such a way that an intermediate space is formed between the seals and is fluidically connected to a coolant and/or lubricant container. The inside diameter of the intermediate space is increased at least in certain areas such that an annular gap or a groove is defined between the piston and the sleeve-like component.

Additional refinements, advantages and possible applications of the invention result from the following description of one embodiment and the FIGURE. In this respect, all described and/or graphically illustrated characteristics form the object of the invention individually or in any combination, independent of their combination in the claims or their reference to other claims.

### BRIEF DESCRIPTION OF DRAWING

FIG. 1 is a schematic section showing the design of one embodiment of a feed pump of this invention.

### DETAILED DESCRIPTION

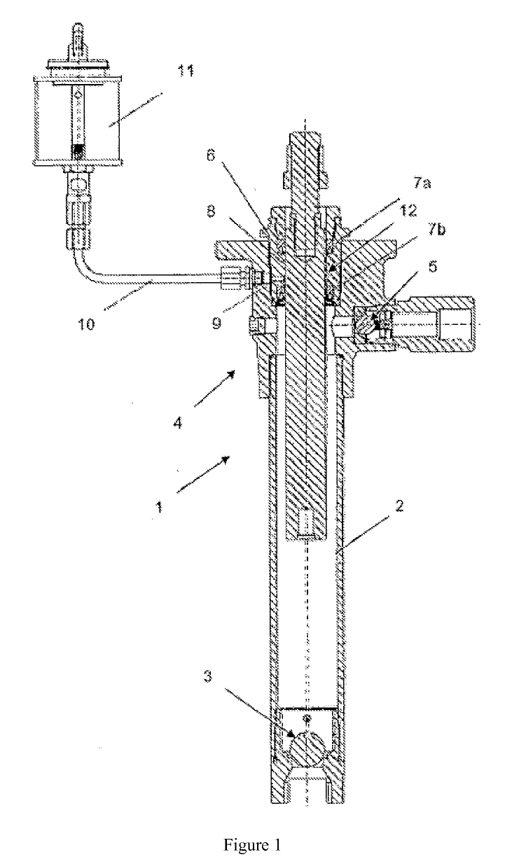

FIG. 1 shows a feed pump 1 comprising a pump housing 2 that is essentially configured as a cylindrical pipe, the lower end of which is closed with an inlet valve 3 that is configured as a check valve. The inlet valve 3 is configured and arranged in such a way that a fluid can flow into the pump housing 2 but not out of the pump housing 2.

The pump housing 2 is sealed at its upper end in FIG. 1 with a connecting head 4. An outlet valve 5 that is also configured as a check valve branches off the connecting head 4 and is configured and arranged in such a way that a fluid can flow out of the pump housing 2 but not into the pump housing 2.

The connecting head 4 further contains a threaded section, into which a screw-in sleeve 6 is screwed in a sealed fashion. Two seals 7a and 7b that are configured as lip seals are arranged in the screw-in sleeve 6 and are spaced apart from one another. A piston 8 is guided in the screw-in sleeve 6 such that it can be displaced in the axial direction of the pump housing 2 and is sealed relative to the surroundings by the two seals 7a and 7b.

The piston 8 can be moved vertically upward from the position illustrated in the FIGURE such that a negative pressure is created in the pump housing 2. This causes the outlet valve 5 to close and the inlet valve 3 to open against the force of a spring or the like such that a medium, for example a UV paint or another high-viscosity medium, is drawn into the pump housing 2. The inlet valve 3 is reclosed when the piston 8 is subsequently moved downward in the FIGURE and shifted into the pump housing 2. The pressure being built up in the pump housing 2 causes the outlet valve 5 to open against the force of a spring or the like such that a medium can be conveyed out of the pump housing 2 through the outlet valve 5.

A bore 9 through the screw-in sleeve 6 and the connecting head 4 is arranged between the two seals 7a and 7b. The bore 9 is connected via a line 10 to a container 11 holding a coolant, lubricant and/or solvent. If applicable, it would also be possible to correspondingly connect several containers to the connecting head 4 and to the screw-in sleeve 6.

The intermediate space inside the screw-in sleeve 6 situated between the two seals 7a and 7b is configured in such a way that the inside diameter of the screw-in sleeve 6 is increased at least in certain areas. In the embodiment illustrated in the FIGURE, a groove 12 is provided on the inside of the screw-in sleeve 6. In this case, the groove 12 is arranged in the screw-in sleeve 6 in such a way that it is connected to the line 10 and therefore to the container 11 via the bore 9.

The movement of the piston 8 creates a slight negative pressure in the intermediate space (gap) between the two seals 7a and 7b, wherein this negative pressure draws in small quantities of a lubricant or the like from the container 11. Consequently, a permanent film of lubricant is applied to the piston 8 in the region of the seals so as to minimize the friction and the temperature of the piston 8.

In addition, the hardening tendency of the media to be pumped can be additionally diminished by utilizing special oils with a solvent additive. This makes it possible to pump UV paints and other media with strong polymerization (hardening) tendencies such as, for example, high-viscosity media, by means of the feed pump 1. The service lives of the seals 7a and 7b used can be extended many times over in this fashion.

Alternatively or additionally to the groove 12 in the screw-in sleeve 6, the intermediate space between the two seals 7a and 7b may also be configured as an annular gap or in another suitable fashion such that a movement of the piston 8 creates a sufficient negative pressure for drawing a medium such as a coolant or lubricant from an external container 11 into the sealing arrangement via a line 10.

It will be observed from the foregoing that the present invention is based on the objective of making available a feed pump and a sealing arrangement therefor which make it possible to pump media with a strong polymerization tendency, particularly UV paints, due to the reduced friction, while simultaneously ensuring an adequate seal and long service lives of the seal.

According to the invention, this objective is essentially attained in that the piston of a feed pump of the initially cited type is guided in at least a section of the pump housing such that it is sealed relative to the surroundings by two seals that are arranged one behind the other in the axial direction of the piston, wherein an intermediate space, particularly a gap-like intermediate space, is formed between the at least two seals and is fluidically connected to a coolant and/or lubricant container.

Due to the connection of the intermediate space situated between the two seals to a coolant and/or lubricant container, a permanent coolant and/or lubricant film that minimizes the friction of the piston in the sealing arrangement forms on the piston due to its movement in the pump housing. The piston and the sealing arrangement are simultaneously cooled due to the supply of a coolant and/or lubricant such that the risk of hardening of the media to be pumped is minimized. The inventive feed pump therefore is particularly suitable for pumping UV paints and similar high-viscosity media, even if they have a strong polymerization tendency. In addition, the lubrication and/or cooling of the sealing arrangement and of the piston make it possible to substantially extend the service life of the seals. Consequently, extended standstill times for exchanging seals can be prevented with the inventive feed pump.

According to one preferred embodiment of the invention, the at least two seals, between which the intermediate space is formed, are configured as lip seals. Alternatively, it would also be possible to provide other suitable seals in the pump housing and/or on the piston.

If the intermediate space of the pump housing situated between the two seals has an increased inside diameter at least in certain areas, an annular gap or a groove is defined between the piston and the pump housing. The coolant and/or lubricant of the coolant and/or lubricant container is able to flow into this annular gap or into this groove to minimize the friction and to lower the temperature of the piston and the sealing arrangement.

According to the invention, it is proposed that the piston and the intermediate space of the pump housing situated between the two seals be configured in such a way that a negative pressure is created in the intermediate space during an axial displacement of the piston. This makes it possible to draw in small quantities of the coolant and/or lubricant from the external container. During the operation of the feed pump, coolant and/or lubricant therefore is automatically supplied to the sealing arrangement and to the piston of the feed pump. Consequently, an additional drive for the lubricant supply can be eliminated.

The connection between the feed pump and a coolant and/or lubricant container is preferably produced by fluidically connecting the intermediate space of the pump housing situated between the at least two seals to the coolant and/or lubricant container by means of a bore or the like in the pump housing and a line connected thereto. Alternatively or additionally, the intermediate space of the pump housing situated between the two seals may also be fluidically connected to a solvent container. Due to this measure, the piston and/or the sealing arrangement is/are wetted with a solvent so as to further lower the risk of the pumped medium hardening. The intermediate space of the pump housing situated between the two seals therefore needs only to be connected to a single container to reduce the friction and the temperature and to simultaneously lower the risk of hardening of the medium to be pumped.

According to one preferred embodiment of the invention, the pump housing essentially consists of a cylindrical pipe that is closed by the inlet valve in the form of a check valve on one end and by a connecting head on the opposite end. This represents a particularly simple design of the pump housing and consequently allows an economical manufacture of the feed pump.

According to an additional refinement of this embodiment, it is proposed that the pump housing feature a connecting head, in which the piston is guided and the outlet valve in the form of a check valve is arranged. In this case, the piston may be guided in a screw-in sleeve of the connecting head, in which the at least two seals are also accommodated. It is therefore possible, if so required, to easily exchange individual components of the feed pump for repair purposes or to adapt these individual components to different operating conditions. The inventive design of the feed pump also allows a cost-efficient manufacture of the pump.

The objective of the invention is further attained with a sealing arrangement that is suitable, in particular, for a feed pump of the initially cited type, and features a piston or the like that is guided in a sleeve-like component. At least two seals are arranged in the sleeve-like component and are spaced apart from one another in such a way that an intermediate space is formed between the seals and is fluidically connected to a coolant and/or lubricant container. The inside diameter of the intermediate space is increased at least in certain areas such that an annular gap or a groove is defined between the piston and the sleeve-like component. This makes it possible to introduce a lubricant, a coolant or the like into the sealing arrangement such that at least the piston is wetted in certain areas. The friction between the piston and the sleeve-like component of the sealing arrangement is minimized in this fashion such that no increased thermal stress occurs due to friction. The service lives of the seals can also be extended due to the reduced friction. The sealing arrangement and the piston may also be additionally cooled with a coolant. It may even be practical to heat rather than cool the piston and the sealing arrangement, depending on the operating conditions.

According to one preferred embodiment of the invention, the piston and the intermediate space of the sleeve-like component of the sealing arrangement situated between the two seals are configured in such a way that at least a slight negative pressure is created in the intermediate space during an axial displacement of the piston. The negative pressure suffices for drawing a medium from the coolant and/or lubricant container into the intermediate space. This makes it possible to automatically convey a coolant and/or lubricant into the sealing arrangement during a movement of the piston.

### LIST OF REFERENCE SYMBOLS

1 Feed pump
2 Pump housing
3 Inlet valve
4 Connecting head
5 Outlet valve
6 Screw-in sleeve
7a, 7b Seal (lip seal)
8 Piston
9 Bore
10 Line
11 (Coolant and/or lubricant) container
12 Groove

1. A feed pump, adapted for pumping UV paints and other high-viscosity media, comprising a pump housing having an inlet valve and an outlet valve, and a piston that is displaceably arranged in the pump housing to pump a medium, wherein the piston is guided in at least a section of the pump housing such that it is sealed relative to the surroundings by at least two seals that are spaced from one another in the axial direction of the piston, and wherein an intermediate space is formed between the at least two seals and is fluidically connected to a coolant and/or lubricant container.

2. The feed pump according to claim 1, characterized by the fact that the two seals are lip seals.

3. The feed pump according to claim 1, characterized by the fact that the intermediate space of the pump housing situated between the two seals has an increased inside diameter at least in certain areas such that an annular gap or a groove is defined between the piston and the pump housing.

4. The feed pump according to claim 3, characterized by the fact that the piston and the intermediate space of the pump housing situated between the two seals are configured in such a way that a negative pressure is created in the intermediate space during an axial displacement of the piston.

5. The feed pump according to claim 4, characterized by the fact that the intermediate space of the pump housing situated between the two seals is fluidically connected to a coolant and/or lubricant container and/or to a solvent container by means of a bore or the like in the pump housing and a line connected thereto.

6. The feed pump according to claim 5, characterized by the fact that the pump housing essentially consists of a cylindrical pipe that is closed by the inlet valve in the form of a check valve on one end and by a connecting head on its opposite end.

7. The feed pump according to claim 6, characterized by the fact that the pump housing comprises a connecting head in which the piston is guided and the outlet valve in the form of a check valve is arranged.

8. The feed pump according to claim 7, characterized by the fact that the piston is guided in a screw-in sleeve of the connecting head, and wherein the seals are accommodated in the screw-in sleeve.

9-10. (canceled)

11. The feed pump according to claim 1, characterized by the fact that the piston and the intermediate space of the pump housing situated between the two seals are configured in such a way that a negative pressure is created in the intermediate space during an axial displacement of the piston.

12. The feed pump according to claim 1, characterized by the fact that the intermediate space of the pump housing situated between the two seals is fluidically connected to a coolant and/or lubricant container and/or to a solvent container by means of a bore or the like in the pump housing and a line connected thereto.

13. The feed pump according to claim 1, characterized by the fact that the pump housing essentially consists of a cylindrical pipe that is closed by the inlet valve in the form of a check valve on one end and by a connecting head on its opposite end.

14. The feed pump according to claim 1, characterized by the fact that the pump housing comprises a connecting head in which the piston is guided and the outlet valve in the form of a check valve is arranged.

15. The feed pump according to claim 1, characterized by the fact that the piston is guided in a screw-in sleeve of the connecting head, and wherein the seals are accommodated in the screw-in sleeve.

16. A sealing arrangement for a feed pump, comprising a piston guided in a sleeve-like component, at least two seals arranged in the sleeve-like component and spaced apart from one another in such a way that an intermediate space is formed between the seals and is fluidically connected to a coolant and/or lubricant container, and wherein the inside diameter of said intermediate space is increased at least in certain areas such that an annular gap or a groove is defined between the piston and the sleeve-like component.

17. The sealing arrangement according to claim 16, characterized by the fact that the piston and the intermediate space of the sleeve-like component situated between the two seals are configured in such a way that a negative pressure is created in the intermediate space during an axial displacement of the piston in order to draw a medium from the coolant and/or lubricant container into the intermediate space.
